(19) United States
(12) Patent Application Publication
(10) Pub. No.: US 2021/0244512 A1
(43) Pub. Date: Aug. 12, 2021

(54) DENTAL IMPLANT

(71) Applicant: Harold J. Clukey, JR., Virginia Beach, VA (US)

(72) Inventor: Harold J. Clukey, JR., Virginia Beach, VA (US)

(21) Appl. No.: 17/168,446

(22) Filed: Feb. 5, 2021

Related U.S. Application Data
(60) Provisional application No. 62/970,856, filed on Feb. 6, 2020.

(51) Int. Cl. A61C 8/00; A61C 8/02; A61L 27/20; A61L 27/34; A61L 27/22; A61L 27/04; A61L 27/54; A61C 13/00; A61B 6/14

(52) U.S. Cl. CPC A61C 8/0037; A61L 2400/12; A61C 8/0006; A61C 8/0013; A61L 27/20; A61L 27/34; A61L 27/227; A61L 27/042; A61L 27/54; A61C 13/0019; A61L 27/047; A61C 8/0075; A61C 13/0004; A61B 6/14; A61C 2008/0046; A61L 2430/12; A61C 8/0036

(57) ABSTRACT

A dental implant and a method of dental restoration. In one embodiment, the dental implant includes at least one root adapted to be inserted into a hole of a mandibular bone or a maxillary bone, and an abutment on top of the root adapted to mate with a crown. Each root may be a lattice scaffold configured to house bone grafting material. A bone grafting material may be inserted within the lattice scaffold to occupy vacant space within the root and the hole of the mandible/maxilla. A method of dental restoration may include obtaining an image of at least a portion of a mandibular bone or a maxillary bone of a patient, drilling a hole into the mandibular bone or maxillary bone, inserting a one-piece dental implant with an abutment and one or more non-threaded roots into the hole, and installing a crown on the abutment of the one-piece dental implant.

### RELATED APPLICATION

The following application claims priority to U.S. Provisional No. 62/970,856, filed Feb. 6, 2020, and incorporated herein by reference in its entirety.

### FIELD OF THE INVENTION

The present invention relates to a dental implant, and in particular, a dental implant that is non-threaded.

### BACKGROUND

While dental care within the United States has general improved, tooth loss due to decay, disease, or injury, remains a problem for many. In some cases, dental implants may be used to replace a lost tooth and root.

A dental implant may be considered as having an anchor or root component suited to osseointegration with the bone tissue within a person's mandible or maxilla, and a prosthetic component or abutment, such as replacement tooth or crown, which engages with or couples to the implant. The implant component (e.g., a titanium screw) is typically fixed within the jaw. As osseointegration fuses the implant to the mandible/maxilla, it provides a stable support for the prosthesis or artificial teeth.

The choice of the type of dental implant surgery to use depends somewhat on the type of implant and the condition of the mandible/maxilla. Endosteel implants are surgically and mechanically affixed or implanted into the mandible/maxilla. Subperiosteal implants involve a metal frame that can be fitted to a mandible/maxilla below a gum tissue line.

Oral surgery to place a dental implant can include cutting open the gum to expose the mandible/maxilla, or soft tissue reflection. With the mandible/maxilla exposed, the surgeon may then drill into the mandible/maxilla to form a hole suitable for receipt of the implant. The implant is then placed into the site, in some cases by mechanical operation of a self-tapping anchor. The mandible/maxilla may then grow bone around the anchor in osseointegration to support the remainder of the dental implant. When secure, the gum may be re-exposed for placement of the abutment to the anchor. In some cases, the abutment may be attached to the implant.

The implants on the market today consists of two-pieces made from titanium. A significant flaw of this design is screw loosening that occurs between the abutment and the implant. Most of the parts for these implants are mass-produced and sold off-the-shelf, thereby limiting the ability for a dentist to customize the implant to the particular needs of a patient. Another problem of importance is the interface between abutments and implants can be lost, and if not handled correctly can lead to screw loosening. Thread designs can also be a problem. Many thread designs do not allow enough space between the threads to allow bone to exist. Also, minimal epithelial connection around an implant abutment allows bacteria to invade around the implant causing failure.

### SUMMARY

To this end, the present invention provides a dental implant with a geometric scaffolding and solid epithelial seal with a firm connection to the bone that adds strength as the bone grafting takes place and prevents bacterial invasion. The dental implant disclosed herein is designed to mimic natural teeth, and preferably comprises one-piece.

Accordingly, one aspect of the present invention is directed to a dental implant comprising at least one root adapted to be inserted into a surgical site of a maxillary bone or mandibular bone, and an abutment on top of the root adapted to mate with a crown. The dental implant may have between 1 to 3 roots. Each root may comprise a lattice scaffold configured to house bone grafting material. The dental implant may further include a crown configured to mate with the abutment.

The dental implant may be adapted to receive a bone grafting material within the lattice scaffold. The bone grafting material occupies vacant space within the root and the hole of the mandible/maxilla. In some embodiments, the bone grafting material may comprise a coated nanomaterial adapted to promote bone growth. Some embodiments of a coated nanomaterial may comprise a sugar-coated nanomaterial. For example, the sugar-coated nanomaterial may comprise a peptide amphiphile scaffold coated with sulfated polysaccharides.

In some embodiments, the lattice scaffold may comprise a metal composite. For example, the metal composite may comprise iron, magnesium and zinc.

The lattice scaffold may comprise a plurality of vertical members joined together by a plurality of struts forming a set of trusses. For example, the struts may be angled to form a set of Warren trusses. In some embodiments, the set of trusses may form an outer wall of the root. The vertical members may be tapered in some embodiments. In some embodiments, the abutment may also comprise a lattice structure.

Another aspect of the present invention is directed to a method of dental restoration. In some embodiments, the method may comprise obtaining an image of at least a portion of a maxillary and mandible bone of a patient, drilling a hole into the maxillary or mandible bone, inserting a one-piece dental implant with an abutment and one or more non-threaded roots into the hole, and installing a crown on the abutment of the one-piece dental implant. The step of obtaining an image may preferably be performed with a computed tomography scanner.

The method may also include the step of 3D printing the one-piece dental implant. For example, the one-piece dental implant may be printed with a metal composite comprising iron, magnesium and zinc. In some embodiments, the method may further comprise filling an internal cavity of the one-piece dental implant with a bone grafting material adapted to promote bone growth.

These and other aspects will become more apparent in view of the drawings and detailed description of the embodiments.

### BRIEF DESCRIPTION OF THE DRAWINGS

FIG. 1 is a side perspective view of a dental implant according to one embodiment;

FIG. 2 is an overhead perspective view of the dental implant in FIG. 1 with a crown mated to the abutment;

FIG. 3 is a side perspective view of a dental implant according to another embodiment;

FIG. 4 is a top perspective view of a mandible with holes drilled in according to one embodiment;

FIG. 5A is a side perspective view of a dental implant having three roots according to another embodiment;

FIG. 5B is a top perspective view of the dental implant in FIG. 5A;

FIG. 5C is a bottom perspective view of the dental implant in FIG. 5A;

FIG. 6A is an enlarged perspective view of a mandible with a hole according to one embodiment;

FIG. 6B is an enlarged perspective view of the mandible in FIG. 6A with a dental implant inserted into the hole;

FIG. 6C is an enlarged perspective view of the mandible in FIG. 6A with a crown mated to the dental implant;

FIG. 7A is a side perspective view of a mandible with a guide mounted on a set of teeth; and

FIG. 7B is a top perspective view of the mandible and guide shown in FIG. 7A.

### DETAILED DESCRIPTION OF THE EMBODIMENTS

The foregoing and other aspects of the present invention will now be described in more detail with respect to the description and methodologies provided herein. It should be appreciated that the invention can be embodied in different forms and should not be construed as limited to the embodiments set forth herein. Rather, these embodiments are provided so that this disclosure will be thorough and complete, and will fully convey the scope of the invention to those skilled in the art.

The terminology used in the description of the invention herein is for the purpose of describing particular embodiments only and is not intended to be limiting of the invention. As used in the description of the embodiments of the invention and the appended claims, the singular forms “a”, “an” and “the” are intended to include the plural forms as well, unless the context clearly indicates otherwise. Also, as used herein, “and/or” refers to and encompasses any and all possible combinations of one or more of the associated listed items.

The term “about,” as used herein when referring to a measurable value such as an amount of a compound, dose, time, temperature, and the like, is meant to encompass variations of 20%, 10%, 5%, 1%, 0.5%, or even 0.1% of the specified amount. Unless otherwise defined, all terms, including technical and scientific terms used in the description, have the same meaning as commonly understood by one of ordinary skill in the art to which this invention belongs.

As used herein, the terms “comprise,” “comprises,” “comprising,” “include,” “includes” and “including” specify the presence of stated features, integers, steps, operations, elements, and/or components, but do not preclude the presence or addition of one or more other features, integers, steps, operations, elements, components, and/or groups thereof.

As used herein, the term “consists essentially of” (and grammatical variants thereof), as applied to the compositions and methods of the present invention, means that the compositions/methods may contain additional components so long as the additional components do not materially alter the composition/method. The term “materially alter,” as applied to a composition/method, refers to an increase or decrease in the effectiveness of the composition/method of at least about 20% or more.

All patents, patent applications and publications referred to herein are incorporated by reference in their entirety. In case of a conflict in terminology, the present specification is controlling.

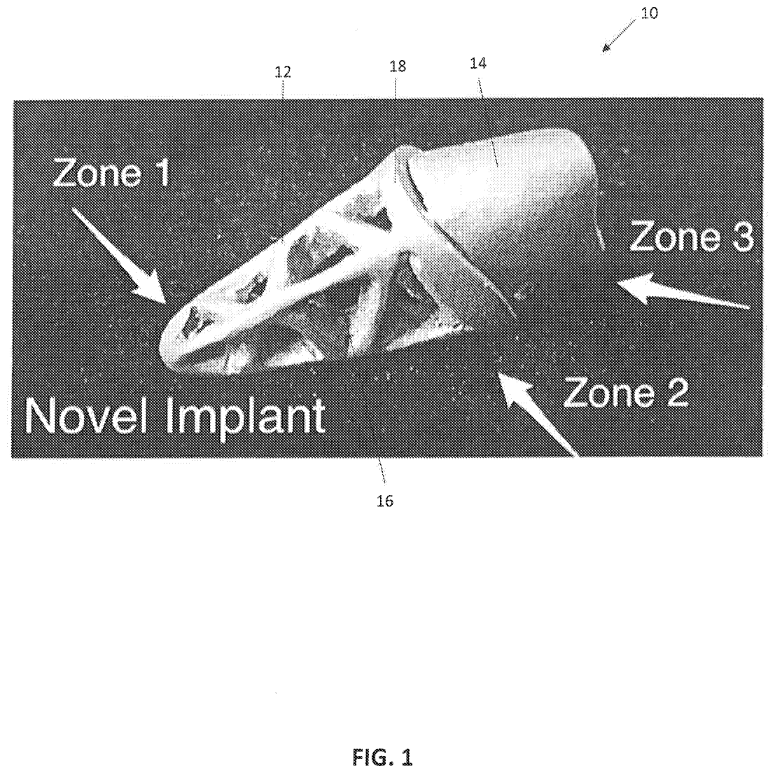

Referring now to FIG. 1, a dental implant 10 is provided. In the embodiment shown, the dental implant 10 comprises a non-threaded root 12 with an abutment 14 on top of the root 12 adapted to mate with a crown. The root 12 has a lattice scaffold 16 providing a reinforced structure to the root, whereas the abutment 14 in this embodiment comprises a solid structure.

The root 12 includes a lip 18 on a top portion that is configured to seal the hole in which the implant 10 is installed. The lip 18 allows space for the attachment of the epithelial tissue and the connective tissue to attach to the dental implant 10. Such lip 18 is absent from other implants. In some embodiments, the lip 18 may be about 2 to 3 mm.

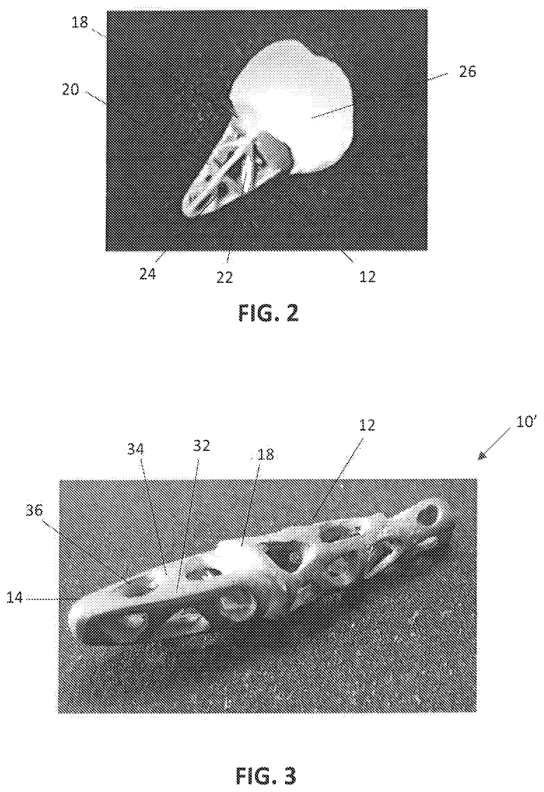

FIG. 2 demonstrates a crown 26 sitting on the lip 18 and mated to the abutment 14. The amount of clearance needed to make a crown 26 may be determined based on the needs of a particular patient. For example, the design of the margin may occur on the abutment, starting with the buccal margin set at 1 mm below biological width, the lingual margin will be set at 0.05 mm below the biological width. In some embodiments, all of these considerations and more may be incorporated into a design software to create a “patient specific” implant.

As seen in FIG. 2, the lattice scaffold 16 may comprise a set of vertical members 20 with struts 22 attached between the vertical members 20 in a variety of configurations to form a set of trusses. For example, the struts 22 may be configured to attached to vertical members 20 at predetermined angles to form a set of Warren trusses. In other embodiments, other truss configurations may be used, including but not limited to sawtooth, Towne, Pratt, and Howe trusses. The vertical members 20 may also be tapered to simulate the shape of a root and/or serve as a wedge to provide a better fit of the dental implant 10 within the hole.

The lattice scaffold 16 may enable bone grafting material to be inserted within the root 12 for bone augmentation. As the bone grafting material is inserted, it fills into the pores 24 of the lattice scaffold 16 and any space within the hole that may be remaining. In some embodiments, the bone grafting material may be a coated nanomaterial adapted to promote bone growth. The coated nanomaterial may comprise a sugar-coated nanomaterial. For example, the sugar-coated nanomaterial may comprise a peptide amphiphile scaffold coated with sulfated polysaccharides as disclosed in Lee et al., “Sulfated glycopeptide nanostructures for multipotent protein activation” Nature Nanotech 12, 821-829 (2017) and incorporated herein by reference in its entirety. Within approximately one year, all of the bone grafting material will be replaced with the patient's own bone.

The dental implant 10 may be made of a metal composite. For example, the dental implant 10 may be made of a biocompatible metal composite comprising iron, magnesium and zinc. While these elements may be toxic at high amounts, the lattice scaffold of the dental implant 10 minimizes the amount used. The metal composite may naturally corrode over time. The combination of this biocompatible metal composite and the bone grafting material results in an osseoinductive effect wherein the dental implant 10 changes from metal to bone and mimics natural tooth formation. Thus in some embodiments, the dental implant 10 may be made of a metal composite comprising iron, magnesium and zinc. The lattice scaffold 16 houses bone grafting material, allowing fusibility of the patient's bone and the bone grafting material. During this period of fusing, the alloys of iron, magnesium and zinc, through corrosion, will be replaced with the patient's own bone. This allows epithelial tissue to form around the “biological width” creating a seal to prevent bacterial invasion.

Other embodiments of the metal composite may include pure titanium (CP—Ti grade 4) and its high strength alloy Ti6A14V (grade 5 or grade 23). The dental implant 10 may preferably be designed and created on-location; for example, within a dentist's or doctor's office. In such embodiments, the dental implant 10 may be created onsite using a 3D printer (i.e., additive manufacturing) using a metal composite. However, it is contemplated that the dental implant 10 may alternatively be manufactured using other means, including subtractive manufacturing and various molding means. The ability to create the dental implant 10 onsite enables the dentist to individually customize the dental implant as needed for a patient, instead of using an off-the-shelf implant with a pre-set design.

In some embodiments, the dental implant 10 may be coated with a medical glue. The medical glue may assist with stabilizing the dental implant 10 within the site of installation and retain it in position. The medical glue may be useful in preventing the dental implant 10 from falling out and enabling grafting to take place. The medical glue may dissolve within a period of three to four weeks.

FIG. 3 depicts an alternative embodiment of a dental implant 10′, wherein the abutment 14 has a lattice structure 30. The lattice structure 30 may comprise a set of vertical members 32 having a plurality of struts 34 forming a set of trusses. The trusses formed include a set of pores 36. The lattice structure 30 enables the dental implant 10′ to accommodate additional bone grafting material, serving as an epithelial attachment.

In some embodiments, the dental implant 10 may comprise more than one root 12 depending on the dental restoration needs of a patient. For example, the dental implant may comprise between one to three roots. The number of roots 12 may vary depending on the site of installation, but it is contemplated that the number of roots may equal the number of roots of the tooth that the dental implant is replacing. For example, all incisors and cuspids have one root and thus a dental implant to replace an incisor or cuspid may comprise one root. Mandibular posteriors have two roots, upper bicuspids have one root, and maxillary molars have three roots. Thus, a dental implant to replace a mandibular posterior tooth may have two roots. Note that it is not required for the dental implant 10 to share the same number of roots 12 as the tooth it is replacing. The dental implant 10 may have more or less roots compared to the original tooth depending on the particular circumstances at hand. Moreover, the dental implant may further include internal struts to further reinforce the structure depending on the size of the implant (e.g., whether additional room is available within the implant).

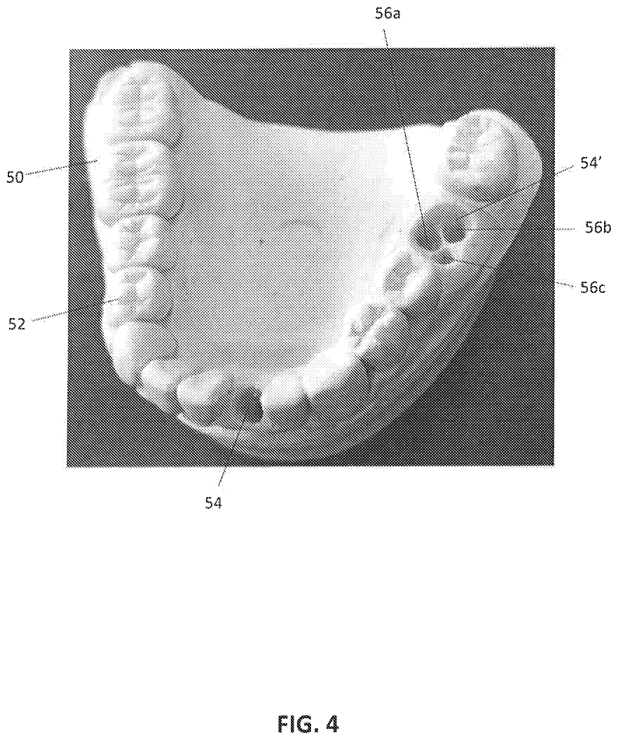

FIG. 4 presents an overview of a lower mandible/maxilla 50 with teeth 52 depicting possible examples of a hole drilled in the mandible/maxilla 50 for inserting a dental implant 10. A single hole 54 may be drilled for installation of a dental implant 10 to replace an incisor. Replacing a lower posterior tooth, such as a molar, may require a hole 54′ having a plurality of subholes 56a, 56b and 56c to accommodate a dental implant having more than one root.

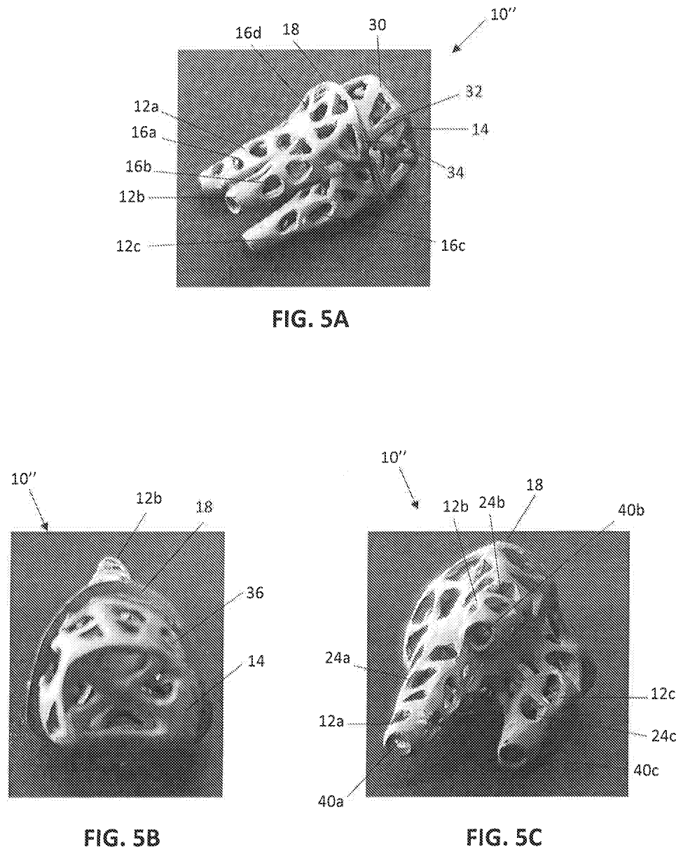

One example of a dental implant having more than one root can be seen in FIGS. 5A-5C. In the example shown, the dental implant 10″ comprises three roots 12a, 12b, and 12c. Each root (12a, 12b, 12c) has its own set of lattice scaffolds (16a, 16b, and 16c, respectively), and may share a portion 16d of the lattice scaffold near the lip 18. Each root (12a, 12b, 12c) comprises a set of vertical members with struts attached between. Moreover, the bottom of each root (12a, 12b, 12c) may further include an additional pore (40a, 40b, and 40c, respectively) adapted for the bone grafting material to fill. The pores 24a, 24b, 24c, 36, 40a, 40b, and 40c found along the dental implant 10″ all serve to expose the bone grafting material along the epithelium and facilitate formation of an epithelial seal to attach the implant. As the dental implant 10″ remains in the hole, the bone grafting material is replaced by the patient's own bone over time. This design results in the strengthening of the implant 10″ over time and prevents the loosening that occurs over time with other dental implants utilizing a screw.

The present invention is also directed to various methods of designing, manufacturing and installing the dental implant 10. Prior to the design, manufacturing and installation, an image of a patient's mandible/maxilla at a point of interest is obtained. Preferably, the image is obtained using a computed tomography (CT) scanner so that the density of bone may be obtained (typically quantified in Hounsfield units). However, images may be obtained by other means including via an X-ray. The dental implant 10 may then be designed and sized based on data from the obtained image. Preferably, the designed dental implant mimics natural teeth with roots (e.g., lower posterior teeth have one to three roots, because of the nerves the root straddles it). In one embodiment, the design and sizing of the dental implant may be performed using computer software. Once the design is finalized, a hole may be drilled into the bone to accommodate the designed implant.

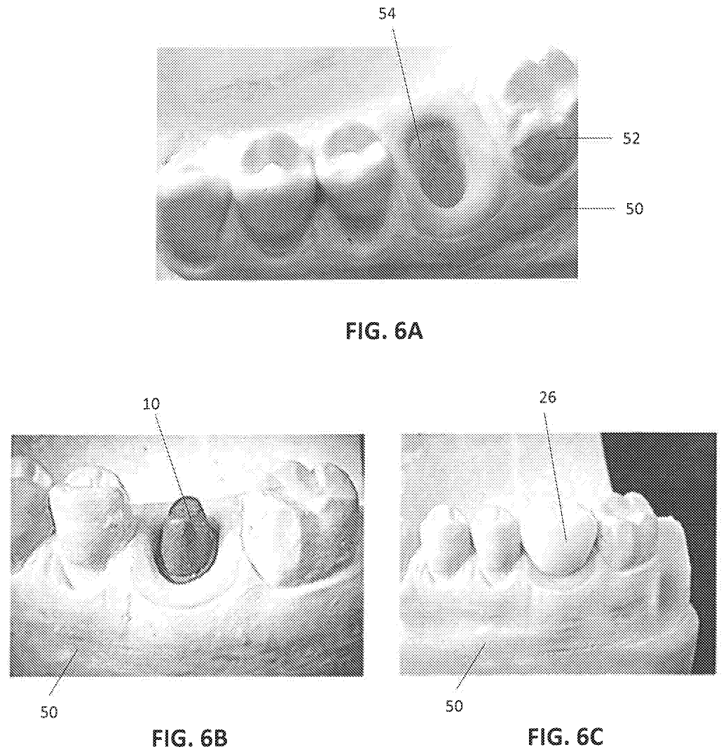

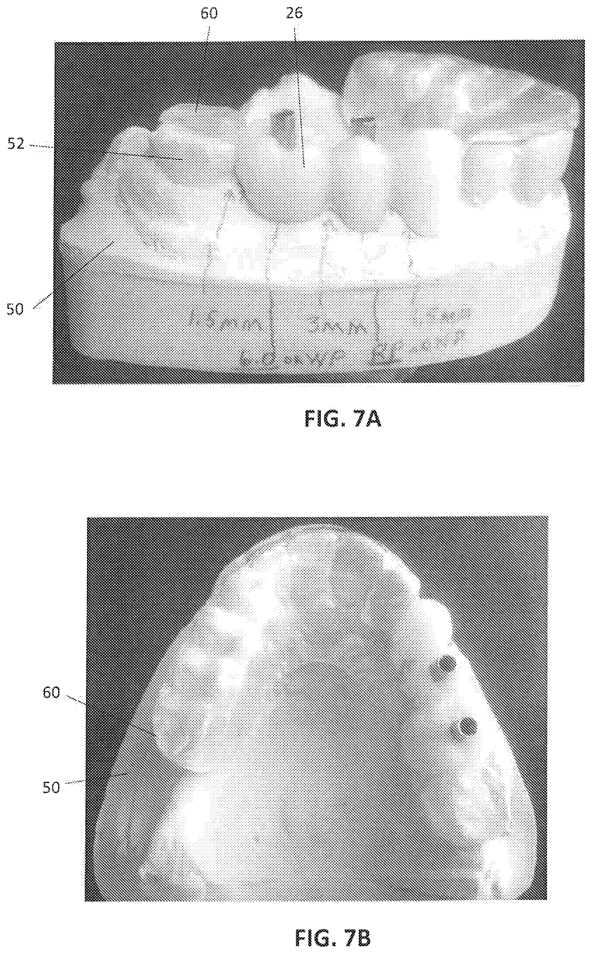

FIGS. 6A-C provides a general overview of the installation of a dental implant 10 into a mandible/maxilla 50 after the imaging and design stages. The mandible/maxilla has a hole 54 drilled at a desired site. The dental implant 10 is then inserted into the hole and bone grafting material is added. The crown 26 is then mated onto the abutment. As seen in FIGS. 7A and 7B, a guide 60 may be used to facilitate installation of one or more dental implants. The guide may be manufactured through various means. For example, the guide may be created from a mold obtained of the patient's mouth. A guide may also be created from a 3D printer and may use data obtained from a CT scan to aid in its design.

Thus, in one embodiment, the present invention may be directed to a method of dental restoration comprising obtaining an image of at least a portion of mandible/maxilla of a patient, drilling a hole into the mandible/maxilla, inserting a one-piece dental implant with an abutment and one or more non-threaded roots into the hole, and installing a crown on the abutment of the one-piece dental implant. In some embodiments, a robot may be used to perform one or more steps of the method. For example, a STL file (or other similar file format) containing design information for the dental implant may be sent to a surgical robot, such as one disclosed by Sun et al., “Automated image-guided surgery for common and complex dental implants, Journal of Medical Engineering & Technology”; 38:5, 251-259 (2014) and Sun et al., “Automated dental implantation using image-guided robotics: registration results” Int J CARS 6, 627-634 (2011), both of which are incorporated herein by reference in their entireties. The surgical robot may prepare the site on the patient's bone and/or insert the implant at the site.

Although the present approach has been illustrated and described herein with reference to preferred embodiments and specific examples thereof, it will be readily apparent to those of ordinary skill in the art that other embodiments and examples may perform similar functions and/or achieve like results. All such equivalent embodiments and examples are within the spirit and scope of the present approach.

That which is claimed:

1. A dental implant comprising:
at least one root comprising a lattice scaffold configured to house bone grafting material and adapted to be inserted into a hole of a mandibular bone or a maxillary bone; and
an abutment on top of the root adapted to mate with a crown.

2. The dental implant of claim 1 further including a bone grafting material within the lattice scaffold configured to occupy vacant space within the root and the hole.

3. The dental implant of claim 2, wherein the bone grafting material comprises a coated nanomaterial adapted to promote bone growth.

4. The dental implant of claim 3, wherein the coated nanomaterial comprises a sugar-coated nanomaterial.

5. The dental implant of claim 4, wherein the sugar-coated nanomaterial comprises a peptide amphiphile scaffold coated with sulfated polysaccharides.

6. The dental implant of claim 1 further including a crown configured to mate with the abutment.

7. The dental implant of claim 1 comprising between one to three roots.

8. The dental implant of claim 1, wherein the lattice scaffold comprises a metal composite.

9. The dental implant of claim 8, wherein the metal composite comprises iron, magnesium and zinc.

10. The dental implant of claim 1, wherein the abutment comprises a lattice structure.

11. The dental implant of claim 1, wherein the abutment and the root are one-piece.

12. The dental implant of claim 1, wherein the lattice scaffold comprises a plurality of vertical members joined together by a plurality of struts forming a set of trusses.

13. The dental implant of claim 12, wherein the struts are angled to form a set of Warren trusses.

14. The dental implant of claim 12, wherein the set of trusses form an outer wall of the root.

15. The dental implant of claim 12, wherein the vertical members are tapered.

16. A method of dental restoration comprising:
obtaining an image of at least a portion of a mandibular bone or a maxillary bone of a patient,
drilling a hole into the mandible/maxilla,
inserting a one-piece dental implant with an abutment and one or more non-threaded roots into the hole, and
installing a crown on the abutment of the one-piece dental implant.

17. The method of claim 14 further including the step of 3D printing the one-piece dental implant.

18. The method of claim 15, wherein the one-piece dental implant is printed with a metal composite comprising iron, magnesium and zinc.

19. The method of claim 14, wherein the step of obtaining an image is performed with a computed tomography scanner.

20. The method of claim 14 further including the step of filling an internal cavity of the one-piece dental implant with a bone grafting material adapted to promote bone growth.
